(19) United States
(12) Patent Application Publication
(10) Pub. No.: US 2011/0208135 A1
(43) Pub. Date: Aug. 25, 2011

(54) INTRAVAGINAL DELIVERY SYSTEM AND PROCESS FOR MANUFACTURING IT

(76) Inventor: Olli Hakala, Turku (FI)

(21) Appl. No.: 13/127,057

(22) PCT Filed: Nov. 16, 2009
(86) PCT No.: PCT/FI2009/050921

§ 371 Date: May 2, 2011

(30) Foreign Application Priority Data
Nov. 19, 2008 (FI) 2008 6093

(51) Int. Cl. A61M 31/00; B29C 45/14

(52) U.S. Cl. 604/285; 156/110.1

(57) ABSTRACT

The present invention concerns an intravaginal delivery system, said system comprising at least one compartment comprising a core and a membrane encasing the core, wherein the core and the membrane essentially consist of a same or different polymer composition. Additionally the intravaginal delivery system comprises a coupling means to form a closed continuous delivery system. The present invention also concerns a method for manufacturing said intravaginal delivery system. The core and/or the membrane are preferably prepared by injection moulding or by extrusion.

The object of the present invention is to provide an intravaginal delivery system, said system comprising at least one compartment comprising a core and a membrane encasing the core, wherein the core and the membrane essentially consist of a same or different polymer composition. Additionally the intravaginal delivery system comprises a coupling means to form a closed continuous delivery system. Another object of the present invention is to provide a method for manufacturing said intravaginal delivery system. The core and/or the membrane are preferably prepared by injection moulding or by extrusion.

### BACKGROUND

Intravaginal delivery systems capable of releasing one or more therapeutically active substances at a substantially constant rate to one another over a prolonged period of time are extremely useful for certain applications, for example contraception and hormone replacement therapy. A number of different constructions of delivery systems, especially of vaginal rings as well as methods to manufacture said systems are known from the literature.

U.S. Pat. No. 3,920,805 describes the manufacture of a solid pharmaceutical device formed as a vaginal ring consisting essentially of a non-medicated central core and an encircling finite thickness of a medicated coating. The method for manufacturing involves (1) placing a siloxane and catalyst mixture into two halves of a mould to provide a so-called centered polymeric core, tightening the mould and curing the siloxane mixture; (2) placing the polymeric core into one half of its mould, filling one half of an outer mould with a medicated polymeric siloxane mixture containing a catalyst, placing the filled one half over the core of the first step and curing; (3) after the mould of the second step is cured filling the other half of the outer mould with the catalysed and medicated polymeric siloxane mixture, bonding this half plus the cured half of the second step together and the whole is cured again. This method requires several manipulations of the ring as it is formed and is relatively labour intensive. Rings made by this method require a trimming of the sprue and of the edges where the two halves of the outer part of the ring are joined, as such edges may cause irritation when the ring has been placed in the body.

U.S. Pat. No. 4,888,074 by DOW CORNING SA provides in one of its aspects a method of making a ring capable of controlled release of a therapeutic agent in the human or animal body. The method includes extruding a composition comprising a therapeutic agent and an elastomer-forming silicone composition to provide a core, extruding a second elastomer-forming silicone composition to provide a sheath to encase the core, bringing together end portions of the extruded core and sheath to form a ring and cross linking the core and the sheath.

The sheath may be extruded onto the core after the latter has been extruded, but is preferably extruded simultaneously with the core. Using this co-extrusion technique the positioning of the core inside the sheath can be sufficiently controlled, keeping it consistent throughout the manufacture. Preferably, the core and the sheath are kept substantially concentric. The extruded core and sheath are of sufficiently cohesive strength to retain their shape. The end portions of the extrudate may be brought together for example by placing the piece in a mould, which has a ring form. The portions are fixed together for example by using a suitable adhesive compound or a layer of uncured elastomer-forming composition, which can crosslink with the second, and preferably with both the first and the second elastomer-forming silicone compositions used in the method.

The intravaginal systems described in U.S. Pat. No. 4,215,691 by ALZA CORP are manufactured by cutting a tubing made of styrene butadiene block copolymer into appropriate lengths, by shaping the piece like a ring and moulding into a torus. Next, a solid polymeric plug having an outside diameter equivalent to the inside diameter of the tube was dampened with methylene chloride and inserted into the tube for joining the opened tube at its two ends, thereby forming a closed system. Then the hollow ring is filled by injecting a steroid carrier mixture into reservoir. Finally, the needle punctures were sealed with a little methylene chloride.

U.S. Pat. No. 4,292,965 by POPULATION COUNCIL describes several methods to manufacture ring formed intravaginal devices. One of the methods consists of forming an annular core ring of suitable inert elastomer in a mould, dipping the ring in a mixture of an inert volatile solvent containing a mixture of contraceptive steroid and an inert unvulcanized elastomer adhesive. Then the solvent is allowed to evaporate and the ring is dipped into a mixture of an inert unvulcanized elastomer in an inert volatile solvent, which is allowed to evaporate to form an outer layer until an outer layer of the desired thickness is obtained. An alternative method of making shell IVRs consists of forming a core in a mould, and allowing it to cure, cutting it into a suitable length, bringing the rod to a constant weight, pulling it through a coating solution containing a mixture of an elastomer and a mixture of estradiol and a progestogen and polymerizing the coating. A membrane is formed by sliding a piece of tubing over the rod. Then a medical grade adhesive is applied to the ends of the rod and to the surface of the rod close to the end. A second piece of swollen tubing of about 4 cm in length is placed over both ends of the rod to form a ring and the ends of the rod are hold together until the adhesive has cured.

In order to control the release rate of therapeutic agents from a ring, it is desirable to have the medication in the core of the ring surrounded by a sheath of non-medicated material. It is of utmost importance to control the centralisation of the core in the ring, in order to ensure the correct release rate. The present invention provides a ring-formed delivery system comprising at least one compartment comprising a core and a membrane, wherein the ends of the core-membrane system are joined together by a coupling means in a way that no impermeable plug is formed at the point of connection.

### BRIEF DESCRIPTION OF THE INVENTION

The present invention concerns an intravaginal delivery system comprising at least one compartment comprising a core of a first cross sectional diameter and a membrane encasing the core, wherein the core and the membrane essentially consist of a same or different polymer composition, forming a core-membrane system with a first end and a first cross sectional surface and a second end and a second cross sectional surface; and a coupling means with a second cross sectional diameter and of a length for connecting the first end and the second end of the core-membrane system, wherein the (second) cross sectional diameter of the coupling means is essentially smaller than the (first) cross sectional diameter of the core.

According to one embodiment of the present invention an adhesive is applied on the coupling means.

According to a further embodiment of the invention an adhesive is applied on the first and/or on the second cross sectional end surface of the core-membrane system. The adhesive can also be applied on the coupling means and on at least one of the cross sectional end surfaces of the core-membrane system.

According to one embodiment of the present invention essentially half of the length of the coupling means is positioned inside the first end of the core-membrane system and rest of the coupling means is positioned inside the second end of the core-membrane system.

According to another embodiment of the present invention there is an opening essentially in the middle of the core in the first and/or in the second end of the core-membrane system, in which opening or openings the coupling means is positioned. The opening can extend through the whole length of the core-membrane system.

According to a further embodiment of the invention the coupling means is a polymeric rod of a biocompatible material.

Intravaginal delivery system according to one embodiment of the present invention comprises the coupling means with a length of 5-25 mm, preferably 10-20 mm.

Intravaginal delivery system of a further embodiment of the invention comprises a core with a cross sectional diameter of 2-10 mm and a coupling means with a cross sectional diameter of 0.5-4.0 mm.

The intravaginal delivery system according to the present invention is suitable for the administration of various types of therapeutically active substances at a predetermined and controlled release rate over a prolonged period of time. The therapeutically active substance can be for example a progestogen or a compound having progestogenic activity or an estrogen or a combination thereof. Additionally the delivery system may comprise at least one other therapeutically active or health promoting substance.

The invention also concerns a method for manufacturing an intravaginal delivery system consisting of at least one compartment comprising a core and a membrane encasing the core, wherein the core and the membrane essentially consist of a same or different polymer composition, the method comprising the steps of forming the core or cores of a first cross sectional diameter, encasing the core or cores by a membrane resulting in a core-membrane system with two ends and connecting the ends of the core-membrane system to form an essentially ring-formed delivery system by a coupling means of a second cross sectional diameter wherein the (second) cross sectional diameter of the coupling means is essentially smaller than the (first) cross sectional diameter of the core or cores.

According to one embodiment of the present invention, the core and/or the membrane are prepared by injection moulding or by extrusion.

### BRIEF DESCRIPTION OF THE FIGURES

FIG. 1 illustrates an exemplary intravaginal delivery system according to the present invention.

FIGS. 2a and 2b illustrate cross sections of the intravaginal delivery system according to the present invention.

FIG. 3 illustrates a preparatory step of the intravaginal delivery system of the present invention.

### DETAILED DESCRIPTION OF THE INVENTION

The present invention concerns an intravaginal delivery system (1) comprising at least one compartment (1a and 1b) comprising a core (2) of a first cross sectional diameter (d₂) and a membrane (3) encasing the core (2), wherein the core (2) and the membrane (3) essentially consist of a same or different polymer composition, forming a core-membrane system (5) with a first end and a second end. The intravaginal delivery system according to the present invention also comprises a coupling means (6) with a second cross sectional diameter (d₆) for connecting the first end and the second end of the core-membrane system (5) to form an essentially ring-formed delivery system (1), in which system the (second) diameter (d₆) of the coupling means (6) is essentially smaller than the (first) diameter (d₂) of the core (2).

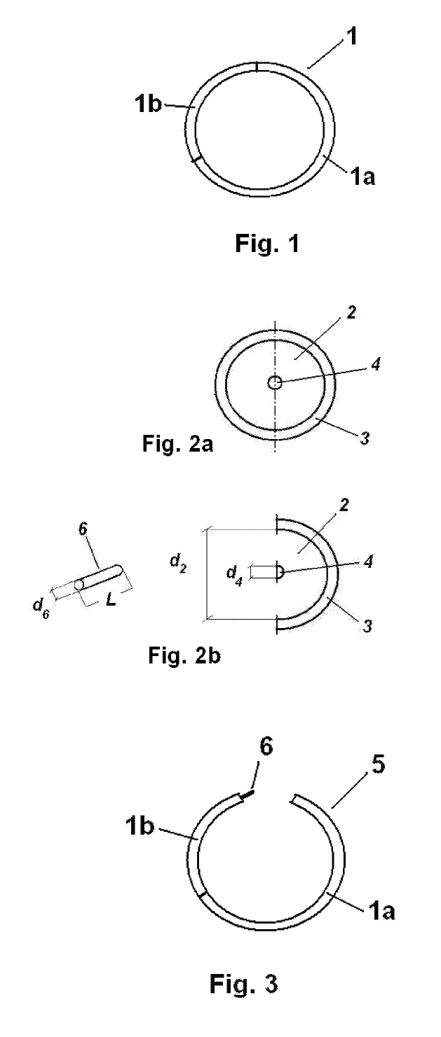

The exemplary intravaginal delivery system (1) shown in FIG. 1 comprises two compartments (1a and 1b) each comprising a core (2) and a membrane (3) encasing said core (2).

In FIG. 2a is shown a cross sectional view of the intravaginal delivery system (1) according to one embodiment of the present invention. The delivery system (1) comprises a core (2), a membrane (3) and an opening (4), which in this specific case is extending through the whole length of the core-membrane system. The cross sectional diameter (d₂) of the core (2) is typically from 2 to 10 mm, preferably between 3.0 to 5.5 mm and more preferably from 4.0 to 5.0 mm.

FIG. 2b indicates the diameters of the intravaginal delivery system shown in FIG. 2a, namely the first cross sectional diameter (d₂) of the core (2), the second cross sectional diameter (d₆) of the coupling means (6) and the cross sectional diameter (d₄) of the opening (4). The length (L) of the coupling means (6) is the distance between its ends.

FIG. 3 illustrates an intravaginal delivery system (1) of the present invention in an open configuration, i.e. the ends of the core-membrane system have not been connected. A core-membrane system (5) comprising two compartments (1a and 1b) each having a core (2) encased by a membrane (3) is manufactured by known methods and cut into a suitable length. A coupling means (6) is partly introduced into the opening (4) at one end of the core-membrane system (5). To form a closed, continuous delivery system (1) as shown in FIG. 1, the other end of the core-membrane system (5) is pulled over the coupling means (6). It is of essential importance that no gap remains in the point of connection i.e. between the ends of the core-membrane system.

The cross sectional diameter (d₆) of the coupling means (6) is from 0.5 to 4.0 mm, typically from 1.0 to 3.0 mm. The length (L) of the coupling means (6) is typically about 5-25 mm, preferably 10-20 mm. On the other hand, if desired and depending on the materials used, in case the opening (4) extends lengthwise through the whole core-membrane system (5), the coupling means (6) can be as long as the core-membrane system. In order to ensure a tight and permanent connection between the ends of the core-membrane system by the coupling means (6), the opening (4) shown in FIG. 2b has a cross sectional diameter (d₄) that is compatible with the cross sectional diameter (d₆) of the coupling means (6). The opening (4) is typically situated in the middle of the core-membrane system as shown in FIG. 2a to ensure the correct release rate. The cross sectional diameter (d₆) of the coupling means (6) can be the same as the cross sectional diameter (d₄) of the opening (4). Depending on the materials used the cross sectional diameter (d₄) of the opening (4) can also be slightly bigger than the cross sectional diameter (d₆) of the coupling means (6). Typically the cross sectional diameter (d₄) of the opening (4) is slightly smaller than the cross sectional diameter (d₆) of the coupling means (6). In some cases it is possible that there is no opening in either end of the core-membrane system (5), i.e. the coupling means (6) is pushed into the core material, preferably into the middle of the core material to ensure the correct release rate.

According to the present invention the coupling means (6) is positioned inside the core-membrane system (5), typically into the opening (4) therein, and it extends from the ends of the core-membrane system (5) a distance therein that imparts both structural support and continuity to the manufactured, closed annular delivery system. Typically about half of the length (L) of the coupling means is situated inside the first end of the core-membrane system (5) and the rest of the coupling means (6) is situated inside the second end of the core-membrane system (5). Thus it is possible that the opening (4) shown in

FIG. 2 extends only a couple of millimeters, typically from 5 to 10 mm from the end of the core-membrane system (5), i.e. the length of the opening (4) within each end of the core-membrane system (5) essentially corresponds to the length of the coupling means inserted into that end. Additionally any other compatible opening (4)—coupling means (6) configuration is also possible and within the scope of the invention as far as the joining of the ends of the core-membrane system is tight and performs seamless continuity between the ends. For example as explained in the description of FIG. 2 the opening (4) can extend lengthwise through the whole core-membrane system (5) and about half of the length (L) of the coupling means having a length (L) of 5-25 mm is inserted into the first end of the core-membrane system (5) and rest of the coupling means (6) is inserted into the second end of the system (5).

In practice, for a human female an outer ring diameter is typically from 35 to 70 mm, preferably from 35 to 58 mm or from 45 to 65 mm and more preferably from 50 to 58 mm. The appropriate length of the core-membrane system can be determined accordingly.

The intravaginal delivery system (1) according to the present invention, i.e. the core (2), the membrane (3) and the coupling means (6), can comprise various materials that are suitable for its intended use and for the intended manufacturing process of the delivery system. The applicable materials are biologically compatible and remain unchanged for a sufficient period of time in the conditions prevailing in the vagina. These materials are known for the skilled person. Examples of suitable materials include, but are not limited to, copolymers of dimethylsiloxanes and methylvinylsiloxanes, ethylene/vinyl acetate copolymers (EVA), polyolefins such as polyethylene and polypropylene, ethylene/propylene copolymers, acrylic acid polymers, ethylene/ethyl acrylate copolymers, polytetrafluoroethylene (PTFE), polyurethanes, polyurethane elastomers, polybutadiene, polyisoprene, poly(methacrylate), polydimethylsiloxane, modified polysiloxanes, for example such as polysiloxane comprising 3,3,3-trifluoropropyl groups attached to the silicon atoms of the siloxane units or comprising poly(alkylene oxide) groups, said poly(alkylene oxide) groups being present as alkoxy-terminated grafts or blocks linked to the polysiloxane units by silicon-carbon bonds, polymethyl methacrylate, styrene-butadiene-styrene block copolymers, polyvinyl chloride, polyvinyl acetate, polyethers, polyacrylonitriles, polyethylene glycols, polymethylpentene and polybutadiene, or a combination of at least two thereof. The preferred material is an elastomer composition comprising poly(alkylene oxide) groups, said poly(alkylene oxide) groups being present as alkoxy-terminated grafts or blocks linked to the polysiloxane units by silicon-carbon bonds, wherein the amount of polydimethylsiloxane comprising poly(alkylene oxide) groups is from 5 to 80 wt-% of the total amount of polymers.

Preferably the coupling means comprise a substantially inert material. The term “substantially inert” means in this connection that the active agent cannot, to any substantial degree, diffuse or in any other way migrate from the core into the coupling means. The coupling means can also be made of a material, which to a certain extent is permeable to the therapeutically active substance(s) of the delivery system.

To ensure a tight connection between the ends of the core-membrane system and to ensure that no gap remains in the point of connection, the coupling can additionally be improved or toughened for example by using solvent bonding, adhesive joining, heat fusing, heat bonding, pressure, and the like. When a solvent is used, the ends of the core-membrane system are moistened with an organic solvent that causes the surfaces to feel tacky, and when placed in contact the surfaces then bond and together with the coupling means adhere in a fluid tight union. The connection between the ends of the core-membrane system can be improved, if necessary, by applying an adhesive or a sealant to at least one end of the system to its cross sectional surface and/or on the surface of the coupling means, and then contacting the ends.

If an adhesive is applied on one or both of the ends of the core-membrane system (5), on their cross sectional surfaces, the adhesive used is preferably permeable to the therapeutically active substances incorporated into the core to ensure that no plug is formed in the point of connection.

The intravaginal delivery system according to the invention is especially suitable for the administration of various types of therapeutically active substances at a predetermined and controlled release rate over a prolonged period of time. The delivery system according to the present invention can comprise a progestogen or a compound having progestogenic activity or an estrogen or a combination thereof as a therapeutically active substance. Additionally the delivery system may comprise at least one other therapeutically active substance, or a health promoting substance capable of giving and/or enhancing the protection against bacterial and fungal infections, and/or enhancing the protection against sexually transmitted diseases.

The intravaginal delivery system according to the present invention consists of at least one compartment comprising a core and a membrane encasing said core, wherein at least one of the cores and/or the membrane, or the surface of the membrane, comprises one or more therapeutically active or a health-promoting substances. Thus, the delivery system may for example consist of one compartment comprising a core and a membrane, wherein the core comprises one or more therapeutically active or health-promoting substances.

Alternatively, the delivery system may consist of at least two compartments, each comprising a core and a membrane encasing said core, wherein at least one of the cores comprises one or more therapeutically active or health-promoting substances. The delivery system may also consist of at least one compartment comprising a core and a membrane encasing said core, wherein at least the membrane or the surface of the membrane comprises one or more therapeutically active or a health-promoting substances.

Any suitable design of the delivery system or any combination of structure is naturally possible and within the scope of the invention.

The invention also concerns a method for manufacturing an intravaginal delivery system consisting of at least one compartment comprising a core and a membrane encasing the core, wherein the core and the membrane essentially consist of a same or different polymer composition, the method comprising the steps of forming the core or cores of a first cross sectional diameter, encasing the core or cores by a membrane resulting in a core-membrane system with two ends and connecting the ends of the core-membrane system to form an essentially ring-formed delivery system by a coupling means of a second cross sectional diameter wherein the (second) diameter of the coupling means is essentially smaller than the (first) diameter of the core or cores.

According to one embodiment of the invention, the core (2) or cores and the membrane (3) are prepared separately, where after the core (2) is encased by the membrane (3).

According to another embodiment of the invention the core (2) or cores and the membrane (3) are prepared simultaneously by using injection moulding or coextrusion.

To manufacture the delivery system a therapeutically active substance is mixed within the polymeric material of the core or the membrane, the mixture is processed to the desired shape by using moulding, injection moulding, extrusion, such as co-extrusion and/or blend-extrusion or other appropriate methods. The membrane can be assembled by mechanically stretching or expanding a prefabricated, tube formed membrane, for example by using pressurised gas, e.g. by air, swelling in a suitable solvent, for example such as cyclohexane, diglyme, propanol, isopropanol or a mixture of solvents, and sliding the expanded membrane tube on the core, or by using extrusion, moulding, spraying or dipping.

The surface of a membrane or one of the membranes can be encased, coated or dusted by particles, crystals, microcrystals, powder or suspension of a therapeutically active or a health-promoting substance. This can be performed by using known methods, for example by spraying the whole delivery system or a part of it with a suspension of said substance in a suitable solvent or by dipping the delivery system in such a suspension.

An especially suitable method for preparing the whole delivery system is disclosed in the Finnish patent FI 97947. This patent discloses an extrusion technology where prefabricated rods containing the active ingredient are coated by an outer membrane. A therapeutically active agent is mixed within the core matrix polymer composition, and processed to the desired shape and size by using known extrusion methods. The membrane layer may then be applied onto the prefabricated cores by feeding the cores to the extruder followed either by another core or a core without any active ingredient, i.e. by a placebo compartment, or by an empty space filled with air, which during the extrusion process will be filled with the membrane material to form a separation membrane. The drug-loaded core and the membrane layer can also be prepared simultaneously by co-extrusion.

The cores or compartments thus obtained can be cut into pieces of the required length and each piece can be assembled in any suitable manner to form a device shaped, sized and adapted for placing in the vagina. The device can have many shapes, for example various continuous, curved shapes, such as annular, ring-shaped, oval, spiral, elliptical, toroidal and the like. The cross section of the device body can have almost any smooth shape, and it can be for example circular, oval, flat, ellipse, star-shaped and the like.

According to the present invention the ends of the core-membrane system are joined together to form a closed continuous delivery system by using a thin polymeric rod as a coupling means to assure a secure and firm bond. The outer diameter of the coupling means is essentially smaller than the diameter of the core. Therefore it does not form an impermeable plug at the point of connection, but allows the diffusion or permeation of the therapeutically active substance(s). If desired, a biocompatible adhesive can be used for better sealing or better adhesion of one end of a core-membrane system to another end or of the coupling means to the compartment.

The delivery system according to the invention can be manufactured in any size as required, the exact size is being dependent on the mammal and particular application. In practice, for human female an outer diameter of the device (the ring) is typically from 35 to 70 mm, preferably from 35 to 58 mm or from 45 to 65 mm. The lengths of the cores of the delivery system are chosen to give the required performance. The length of the drug containing compartment can be for example from 5 mm to 160 mm, or up to the total length of the delivery system.

The intravaginal delivery system made in accordance with the invention can be sterilized by using known methods, for example by using heat, ethylene oxide or radiation.

### Example 1

### A General Method for Manufacturing an Intravaginal Delivery System

99.3 parts of commercial poly(dimethylsiloxane-co-vinylmethylsiloxane), 0.4 parts of poly(hydrogenmethylsiloxane-co-dimethylsiloxane) crosslinker, 0.05 parts of ethynyl cyclohexanol inhibitor and 0.2 parts of Pt-catalyst (of the reaction species) in vinyl-methyl-siloxane were mixed in a kneating mill. The mixture is extruded to a tube-like form, having inner diameter of 1.1 mm and outer diameter of 3.9 mm, and cured by heat at +115° C. for 30 minutes and cooled.

Membrane is prepared by mixing 50 parts of a mixture consisting of 72.3 parts of commercial vinyl terminated poly(dimethylsiloxane-co-vinylmethylsiloxane), 25.5 parts of a silica filler, 0.1 part of ethynylcyclohexanol, 2 parts of poly(hydrogenmethyl-siloxane-co-dimethylsiloxane) crosslinker and 0.05 part of α-tocopherol as a cocatalyst, and 50 parts of a mixture consisting of 97.5 parts of commercial vinyl terminated poly(dimethylsiloxane-co-vinylmethylsiloxane), 0.1 part of ethynylcyclohexanol, 2 parts of poly(hydrogenmethylsiloxane-co-dimethylsiloxane) crosslinker and 0.3 parts of Pt-catalyst (of the reaction species) in vinyl-methyl-siloxane. The mixtures are mixed separately.

The membrane material is combined and coating extruded on the above prepared core and shock cured to yield a tube-formed rod having an outer diameter of 4.4 mm.

### Example 2

### Manufacture of a Delivery System Comprising Drospirenone and Estradiol

An intravaginal delivery system comprising drospirenone and estradiol valerate is prepared. The first core comprising drospirenone (30 wt-%) consists of PEO-b-PDMS (18 wt-% of the total polymer amount) and PDMS and the length of the core is 130 mm. The second core comprising estradiol valerate (15 wt-%) consists of PEO-b-PDMS (15 wt-% of the total polymer amount) and PDMS, and the length is 40 mm. The outer diameter of the cores are 3.9 mm. The core parts are encased in a membrane consisting of PEO-b-PDMS/PDMS in a ratio of 20:80. The membrane layer is applied onto the prefabricated cores by using coextrusion. An empty space of 3 mm left between the drug containing cores is during the process filled by the membrane material thus forming a separation membrane between the cores. The thickness of the membrane wall is 0.4 mm, and the outer diameter of the core-membrane system is 4.7 mm. A 10 mm long polyethylene rod having an outer diameter of 1.2 mm is used as a coupling means. An adhesive (Nusil Med 1-4213) is spread on the cross sectional surface of the other end of the core-membrane system and on the other end of the coupling means, which is then pushed approximately 5 mm into the core. Now the cross sectional surface of the other end of the core-membrane system is glued by using the same adhesive and pushed over the glued polyethylene rod so that the ends of the core-membrane system meet each other. The adhesive is cured at 100° C. for 1 hour.

### Example 3

### Manufacture of a Delivery System Comprising Drospirenone, Ethinyl Estradiol and Lactobacillus

An intravaginal delivery system comprising drospirenone, ethinyl estradiol and Lactobacillus rhamnosus is prepared. The first core comprising drospirenone (30 wt-%) consists of PEO-b-PDMS (45 wt-% of the total polymer amount) and PDMS, and the length of the core is 140 mm. The second core comprising ethinyl estradiol (10 wt-%) consist of PEO-b-PDMS (15 wt-% of the total polymer amount) and PDMS, and the length of the core is 20 mm. An inert placebo core of 10 mm consisting of PDMS is added between the drug containing cores to separate them. All cores are prepared by extrusion to yield a tube-like cores having the outer diameter of 3.8 mm and the inner diameter of 1.1 mm.

The core parts are encased in a membrane consisting of PEO-b-PDMS/PDMS in a ratio of 20:80. The thickness of the membrane wall is 0.3 mm, the inner diameter of the tube is 3.7-3.75 mm and the outer diameter is 4.3-4.35 mm. The ends of the delivery system are joined together into a closed system by using a 10 mm long polyethylene rod having outer diameter of 1.2 mm as a coupling means. An adhesive (Nusil Med 1-4213) is spread on the other end of the coupling means and polyethylene rod is pushed approximately 5 mm into the core. The cross sectional surfaces of the core-membrane tube and the other end of the coupling means are dabbed with the same adhesive and the other end of the core-membrane system is pushed over the polyethylene rod so that the ends of the core-membrane system meet each other. The adhesive is cured at 100° C. for 1 hour. Finally the delivery system is dipped in the suspension of Lactobacillus rhamnosus in a hard fat (Witepsol®) to give a thin coating.

### Example 4

### Manufacture of a Delivery System Comprising Drospirenone

An intravaginal delivery system comprising drospirenone is prepared. A core comprising drospirenone (30 wt-%) consists of PEO-b-PDMS (45 wt-% of the total polymer amount) and PDMS, and the length of the core is 167 mm. The core is prepared by extrusion to yield a tube-like core having the outer diameter of 4.1 mm and the inner diameter of 1.1 mm.

The core is encased in a membrane consisting of PEO-b-PDMS/PDMS in a ratio of 20:80. The thickness of the membrane wall is 0.4 mm, the inner diameter of the tube is 4.05 mm and the outer diameter is 4.85 mm. The ends of the delivery system are joined together into a closed system by using a 12 mm long polyethylene rod having outer diameter of 1.1 mm as a coupling means. An adhesive (Nusil Med 1-4213) is spread on the other end of the coupling means and polyethylene rod is pushed approximately 6 mm into the core. The cross sectional surfaces of the core-membrane tube and the other end of the coupling means are dabbed with the same adhesive and the other end of the core-membrane system is pushed over the polyethylene rod so that the ends of the core-membrane system meet each other. The adhesive is cured at 100° C. for 1 hour.

1. Intravaginal delivery system (1) comprising
at least one compartment (1a, 1b) comprising a core (2) of a first cross sectional diameter (d₂) and a membrane (3) encasing the core (2), wherein the core (2) and the membrane (3) essentially consist of a same or different polymer composition, forming a core-membrane system (5) with a first end and a first cross sectional surface and a second end and a second cross sectional surface and
a coupling means (6) with a second cross sectional diameter (d₆) and of a length (L) for connecting the first end and the second end of the core-membrane system (5)
characterized in that the (second) cross sectional diameter (d₆) of the coupling means (6) is essentially smaller than the (first) cross sectional diameter (d₂) of the core (2).

2. Intravaginal delivery system (1) according to claim 1, wherein an adhesive is applied on the coupling means (6).

3. Intravaginal delivery system (1) according to claim 1 or 2, wherein an adhesive is applied on the first and/or the second cross sectional end surface of the core-membrane system (5).

4. Intravaginal delivery system (1) according to claim 1, wherein essentially half of the length (L) of the coupling means is positioned inside the first end of the delivery system and rest of the coupling means is positioned inside the second end of the delivery system.

5. Intravaginal delivery system (1) according to claim 4, wherein there is an opening (4) essentially in the middle of the core (2) in the first end and/or in the second end of the core-membrane system (5), in which opening (4) or openings the coupling means (6) is positioned.

6. Intravaginal delivery system (1) according to claim 4, wherein there is an opening (4) essentially in the middle of the core (2), the opening (4) extending through the whole length of the core-membrane system (5), and in which opening (4) the coupling means (6) is positioned.

7. Intravaginal delivery system (1) according to claim 1, wherein the coupling means (6) is a polymeric rod of a biocompatible material.

8. Intravaginal delivery system (1) according to claim 1, wherein the length (L) of the coupling means (6) is 5-25 mm, preferably 10-20 mm.

9. Intravaginal delivery system (1) according to claim 1, wherein the cross sectional diameter (d₂) of the core (2) is 2-10 mm and the cross sectional diameter (d₆) of the coupling means (6) is 0.5-4.0 mm.

10. Intravaginal delivery system (1) according to claim 1 suitable for administration of various types of therapeutically active substances at a predetermined and controlled release rate over a prolonged period of time.

11. Intravaginal delivery system (1) according to claim 10 wherein the therapeutically active substance is a progestogen or a compound having progestogenic activity, or an estrogen or a combination thereof.

12. Intravaginal delivery system (1) according to claim 11 wherein at least one other therapeutically active or health promoting substance is present.

13. Method for manufacturing an intravaginal delivery system (1) consisting of at least one compartment (1a, 1b) comprising a core (2) and a membrane (3) encasing the core (2), wherein the core (2) and the membrane (3) essentially consist of a same or different polymer composition, the method comprising the steps of
forming the core (2) or cores of a first cross sectional diameter (d₂)
encasing the core (2) or cores by a membrane (3) resulting in a core-membrane system (5) with two ends and
connecting the ends of the core-membrane system (5) to form an essentially ring-formed delivery system (1) by a coupling means (6) of a second cross sectional diameter (d₆)
characterized in that the (second) cross sectional diameter (d₆) of the coupling means (6) is essentially smaller than the (first) cross sectional diameter (d₂) of the core (2) or cores.

14. Method for manufacturing an intravaginal delivery system according to claim 13, wherein the core (2) or cores and/or the membrane (3) are prepared by injection moulding or by extrusion.
